(19) United States
(12) Patent Application Publication
(10) Pub. No.: US 2025/0295660 A1
(43) Pub. Date: Sep. 25, 2025

(54) METHODS OF TREATING NF1-MUTANT TUMORS USING LSD1 INHIBITORS

(71) Applicant: ORYZON GENOMICS, S.A., Madrid (ES)

(72) Inventors: Robert SOLIVA SOLIVA, Sant Feliu del Llobregat (ES); Natalia SACILOTTO, Cornellà de Llobregat (ES); Cristina MASCARÓ CRUSAT, Cornellà de Llobregat (ES); David ROTLLANT POZO, Cornellà de Llobregat (ES)

(73) Assignee: ORYZON GENOMICS, S.A., Madrid (ES)

(21) Appl. No.: 18/863,581

(22) PCT Filed: May 9, 2023
(86) PCT No.: PCT/EP2023/062283

§ 371 Date: Nov. 6, 2024

(30) Foreign Application Priority Data
May 9, 2022 (EP) 22382443.4
May 9, 2022 (EP) 22382444.2

(51) Int. Cl. A61K 31/506; A61K 31/135; A61K 31/496; A61P 35/00

(52) U.S. Cl. CPC A61K 31/506; A61K 31/135; A61K 31/496; A61P 35/00

(57) ABSTRACT

The present invention relates to an LSD1 inhibitor for use in the treatment of an NF1-mutant tumor, i.e. a tumor having one or more mutations or genetic alterations affecting the NF1 gene. The invention likewise provides methods of treating an NF1-mutant tumor in a subject in need thereof, comprising administering a therapeutically effective amount of an LSD1 inhibitor to the subject.

### FIELD

The present invention relates to the field of therapy of NF1-mutant tumors, i.e. tumors having one or more mutations or genetic alterations affecting the NF1 gene. In particular, the invention provides an LSD1 inhibitor for use in the treatment of an NF1-mutant tumor. The invention likewise provides methods of treating an NF1-mutant tumor in a subject in need thereof, comprising administering a therapeutically effective amount of an LSD1 inhibitor to the subject.

### BACKGROUND

Tumors, particularly malignant tumors (cancer), are one of the leading causes of death worldwide. The way cancer is clinically managed has radically changed over the last decades. In the past, cancer could only be treated with chemotherapeutic “dirty” drugs, which unspecifically limit cell proliferation by interfering with DNA or the basic cell-cycle machinery. Nowadays, new personalized and precision medicine approaches are instead designed to block the growth of cancer cells while mostly sparing other cells of the body. The advantage of this approach is that targeted therapies are less harmful to normal cells.

Epigenetics is one of the emerging fields in cancer precision medicine, with a first generation of drugs reaching FDA-approval and many more progressing in clinical trials. Lysine specific demethylase 1 (LSD1 or KDM1A) is an epigenetic enzyme regulating gene expression by demethylating the histone H3 tail on two different residues, i.e. lysine 4 (H3K4) and lysine 9 (H3K9), with opposing effects. Demethylation of H3K4 is associated with transcriptional repression whereas demethylation of H3K9 is associated with transcriptional activation. Additionally, LSD1 is part of many multiprotein complexes controlling enhancer-promoter contacts involved in gene repression such as NurD and CoRest. LSD1 has been shown to play a key role in cancers such as leukemia and small cell lung cancer (SCLC), and huge efforts have been committed to the development of LSD1 inhibitors. Catalytic active-site targeted inhibitors have been developed such as iadademstat, bomedemstat and pulrodemstat. These compounds bind deep in the active site of LSD1, blocking access to both protein substrates (such as the histone H3 tail) and non-substrate protein interactors (such as SNAG-domain transcription factors), thereby inhibiting both LSD1 catalytic activity and scaffolding interactions. LSD1 inhibitors have been described to have highly potent anti-proliferative activity in specific tumor types and are currently being tested in clinical trials as treatment for cancers such as acute myeloid leukemia (AML) and SCLC.

NF1 is a tumor suppressor gene that is mutated in neurofibromatosis type I, one of the most common monogenic conditions, affecting about 1 in every 3000 people. It is an autosomal dominant syndrome, characterized by predisposing patients to developing benign tumors called plexiform neurofibromas (PN) which can ultimately become malignant (malignant peripheral nerve sheath tumors-MPNST) and to a range of concomitant neurodevelopmental problems such as cognitive and learning disabilities, epilepsy, problems of speech, autism and hyperactivity. Neurofibromatosis patients are also prone to developing a range of malignant tumors other than MPNST, such as gliomas, gastrointestinal stromal tumors, rhabdomyosarcomas and leukemias. The NF1 gene, as many other tumor suppressors such as p53, PTEN or Rb, is also frequently mutated in a wide variety of non-neurofibromatosis associated cancers. Thus, it has been described that somatic mutations of the NF1 gene can have a frequency as high as 12-30% for cutaneous melanoma, 3.5-23.6% for acute myeloid leukemia, 12-34.4% for ovarian carcinoma, 14-23% for glioblastoma, and 10.3-11% for lung squamous cell carcinoma, among others (Philpott C et al., Hum Genomics, 2017, 11 (1): 13, doi: 10.1186/s40246-017-0109-3). Therefore, mutations in the NF1 gene contribute to the development of a wide range of malignancies, both with and without neurofibromatosis type I. The NF1 gene protein product, neurofibromin, is a negative regulator of the RAS pathway, a signaling axis that promotes cell growth and division. An accepted hypothesis is that germline or somatic mutations of the NF1 gene contribute to the progression of cancer via impairing the negative control neurofibromin exerts on RAS (Philpott C et al., loc. cit.; Tao J et al., Cancer Cell Int, 2020, 20:492, doi: 10.1186/s12935-020-01570-8). However, NF1 is a huge gene consisting of about 60 exons, which codes for a large multi-domain protein consisting of over 2800 amino acids. Thus, it might have many functions other than RAS inhibition which might also be relevant in oncogenesis. Many malignant tumors driven in part by NF1 gene mutations have very poor prognosis and are still in need of targeted treatments. Thus, the development of new therapeutic options to treat NF1-mutant tumors is of great pharmaceutical and medical interest. The present invention addresses this and other needs.

### SUMMARY OF THE INVENTION

The present invention is based on the surprising finding that LSD1 inhibitors, such as e.g. iadademstat, bomedemstat and pulrodemstat, are advantageously effective in the treatment of NF1-mutant tumors, including in particular NF1-mutant acute myeloid leukemia (AML), NF1-mutant acute lymphocytic leukemia (ALL), NF1-mutant malignant rhabdoid tumor (MRT), NF1-mutant small-cell lung cancer (SCLC), NF1-mutant malignant peripheral nerve sheath tumor (MPNST), and NF1-mutant plexiform neurofibroma, as also described in the examples section further below. The present invention thus provides a particularly advantageous and targeted therapeutic approach for the treatment of NF1-mutant tumors.

Accordingly, the present invention relates to an LSD1 inhibitor for use in the treatment of an NF1-mutant tumor. The invention also relates to a pharmaceutical composition comprising an LSD1 inhibitor and optionally one or more pharmaceutically acceptable excipients for use in the treatment of an NF1-mutant tumor.

The invention likewise provides a method of treating an NF1-mutant tumor in a subject in need thereof, comprising administering a therapeutically effective amount of an LSD1 inhibitor (or a therapeutically effective amount of a pharmaceutical composition comprising an LSD1 inhibitor and optionally one or more pharmaceutically acceptable excipients) to the subject.

Moreover, the invention relates to the use of an LSD1 inhibitor for the treatment of an NF1-mutant tumor.

The invention further relates to the use of an LSD1 inhibitor for the preparation of a medicament (or a pharmaceutical composition) for the treatment of an NF1-mutant tumor.

### DETAILED DESCRIPTION OF THE INVENTION

As explained above, the present invention is based on the surprising finding that LSD1 inhibitors are advantageously effective in the treatment of NF1-mutant tumors, including in particular NF1-mutant acute myeloid leukemia (AML), NF1-mutant acute lymphocytic leukemia (ALL), NF1-mutant malignant rhabdoid tumor (MRT), NF1-mutant small-cell lung cancer (SCLC), NF1-mutant malignant peripheral nerve sheath tumor (MPNST), and NF1-mutant plexiform neurofibroma, as also described and demonstrated in the examples section further below. Thus, the exemplary LSD1 inhibitor iadademstat was found to be highly effective in a range of different NF1-mutant tumor cell lines. Further LSD1 inhibitors, having different chemical scaffolds and including both reversible and irreversible inhibitors of LSD1, were likewise confirmed to be effective in the treatment of NF1-mutant tumors, as also described in the examples section.

The present invention thus relates to an LSD1 inhibitor for use in the treatment of an NF1-mutant tumor. The invention also relates to a pharmaceutical composition comprising an LSD1 inhibitor and optionally one or more pharmaceutically acceptable excipients for use in the treatment of an NF1-mutant tumor. The invention likewise provides a method of treating an NF1-mutant tumor in a subject in need thereof, comprising administering a therapeutically effective amount of an LSD1 inhibitor (or a therapeutically effective amount of a pharmaceutical composition comprising an LSD1 inhibitor and optionally one or more pharmaceutically acceptable excipients) to the subject. Moreover, the invention relates to the use of an LSD1 inhibitor for the treatment of an NF1-mutant tumor. The invention further relates to the use of an LSD1 inhibitor for the preparation of a medicament (or a pharmaceutical composition) for the treatment of an NF1-mutant tumor.

In accordance with the present invention, an “LSD1 inhibitor” refers to a compound that reduces, decreases, blocks or inhibits the gene expression, activity or function of LSD1. Examples thereof are provided below under the heading “LSD1 inhibitors”. Preferred LSD1 inhibitors include each one of iadademstat or a pharmaceutically acceptable salt thereof (e.g., iadademstat dihydrochloride), pulrodemstat or a pharmaceutically acceptable salt thereof (e.g., pulrodemstat besylate), and bomedemstat or a pharmaceutically acceptable salt thereof (e.g., bomedemstat bis-tosylate). A particularly preferred LSD1 inhibitor is iadademstat or a pharmaceutically acceptable salt thereof (e.g., iadademstat dihydrochloride).

The LSD1 inhibitor (e.g., iadademstat or a pharmaceutically acceptable salt thereof) is preferably administered orally. Exemplary formulations which can be administered orally, particularly via peroral ingestion, are described in more detail further below.

The subject to be treated in accordance with the invention may be a human being or an animal (e.g., a non-human mammal), and is preferably a human.

The NF1-mutant tumor to be treated in accordance with the present invention may be any tumor (including, e.g., any one of the specific types of tumor listed further below) having one or more mutations or genetic alterations affecting the NF1 gene (e.g., any one or more of the specific mutations/genetic alterations mentioned or referenced herein below), particularly one or more inactivating mutations or inactivating genetic alterations affecting the NF1 gene. Accordingly, the invention particularly relates to the treatment of an NF1-mutant tumor having one or more inactivating mutations or inactivating genetic alterations in the NF1 gene. Such inactivating mutations or inactivating genetic alterations affecting the NF1 gene particularly include loss-of-function mutations and lead to a decrease or absence of expression and/or stability and/or activity of its protein product neurofibromin. Moreover, such mutations or genetic alterations may affect one or both alleles of the NF1 gene.

The NF1-mutant tumor to be treated in accordance with the invention may be malignant (cancerous) or benign (non-cancerous). Moreover, the malignant or benign NF1-mutant tumor may be solid or non-solid. It is preferred that the NF1-mutant tumor is an NF1-mutant malignant tumor, i.e. an NF1-mutant cancer.

Examples of an NF1-mutant malignant tumor (or NF1-mutant cancer) to be treated in accordance with the invention include, in particular, NF1-mutant leukemia (e.g., NF1-mutant acute myeloid leukemia (AML), NF1-mutant acute lymphocytic leukemia (ALL), or NF1-mutant T-cell acute lymphoblastic leukemia), NF1-mutant lymphoma (e.g., NF1-mutant non-Hodgkin lymphoma, including also NF1-mutant Burkitt's lymphoma), NF1-mutant lung cancer (e.g., NF1-mutant small-cell lung cancer (SCLC) or NF1-mutant non-small-cell lung cancer (NSCLC), including also NF1-mutant lung squamous cell carcinoma), NF1-mutant breast cancer (e.g., NF1-mutant triple-negative breast cancer), NF1-mutant esophagogastric cancer, NF1-mutant esophageal cancer, NF1-mutant gastric cancer, NF1-mutant gastrointestinal cancer, NF1-mutant colorectal cancer (e.g., NF1-mutant colorectal carcinoma), NF1-mutant liver cancer, NF1-mutant ovarian cancer, NF1-mutant uterine cancer (e.g., NF1-mutant uterine carcinoma), NF1-mutant cervical cancer (e.g., NF1-mutant cervical carcinoma), NF1-mutant pancreatic cancer, NF1-mutant prostate cancer (e.g., NF1-mutant prostate adenocarcinoma), NF1-mutant bladder cancer (e.g., NF1-mutant bladder carcinoma), NF1-mutant pheochromocytoma, NF1-mutant head and neck cancer (e.g., NF1-mutant head and neck carcinoma), NF1-mutant neuroblastoma, NF1-mutant glioblastoma, NF1-mutant optic pathway glioma, NF1-mutant skin cancer (e.g., NF1-mutant melanoma, including also NF1-mutant desmoplastic melanoma), NF1-mutant malignant rhabdoid tumor, NF1-mutant rhabdomyosarcoma, NF1-mutant Ewing sarcoma, or NF1-mutant malignant peripheral nerve sheath tumor (MPNST).

As explained above, while the NF1-mutant tumor to be treated is preferably an NF1-mutant malignant tumor, the present invention also specifically relates to the treatment of NF1-mutant benign tumors which likewise constitute pathological conditions (and may also be referred to as NF1-mutant benign tumorous disorders). Examples of an NF1-mutant benign tumor include, in particular, NF1-mutant plexiform neurofibroma, NF1-mutant ganglioneuroma, NF1-mutant Lisch nodule, or NF1-mutant cutaneous neurofibroma. The NF1-mutant benign tumor may also be an NF1-mutant premalignant tumor.

Particularly preferred examples of NF1-mutant tumors to be treated in accordance with the present invention include NF1-mutant leukemia (e.g., NF1-mutant AML or NF1-mutant ALL), NF1-mutant malignant rhabdoid tumor, NF1-mutant lung cancer (e.g., NF1-mutant SCLC), NF1-mutant MPNST, or NF1-mutant plexiform neurofibroma.

Furthermore, the NF1-mutant tumor to be treated may also be NF1-mutant tumor which is not NF1-mutant MPNST (including the aforementioned specific types of malignant or benign NF1-mutant tumors which are different from MPNST).

In humans, the NF1 gene is located on chromosome 17q11.2 and codes for a protein product called neurofibromin. The canonical amino acid sequence of isoform 2 of human neurofibromin is 2839 residues long (see, e.g., Uniprot identifier P21359-1; https://www.uniprot.org/uniprot/P21359.fasta), and that of isoform 1 is 2818 residues long (see, e.g., Uniprot identifier P21359-2; https://www.uniprot.org/uniprot/P21359-2.fasta). These two isoforms are considered the most biologically relevant. Isoform 2 is found in most human tissues but is not present in neurons of the central nervous system. The NF1 gene is capable of generating other alternatively spliced isoforms by different combinations of its about 60 exons.

More than 3000 germline mutations in the NF1 gene have been reported in the Human Gene Mutation Database (HGMD; http://www.hgmd.cf.ac.uk/ac/index.php) and more than 1000 somatic mutations in The Cancer Genome Atlas (TCGA; https://www.cancer.gov/about-nci/organization/ccg/research/structural-genomics/tcga); see also Scheer M et al., Int J Mol Sci, 2021, 23 (1): 352, doi: 10.3390/ijms23010352. Many of the characterized mutations are loss-of-function, meaning they ultimately have a negative impact on the functionality of the protein product. At the gene level, all sorts of mutations have been described, including nonsense, missense, frameshift, indels (insertions or deletions), microdeletions, inversions, splice site variants, whole translocations and complex re-arrangements. However, there is no clear pattern of localized mutational clustering within the NF1 gene. In order to identify mutations or genetic alterations in the NF1 gene, it is advisable to apply a mutation detection pipeline to characterize NF1 mutants reliably, where a series of different algorithms specifically fine-tuned to detect single nucleotide variants (SNVs), indels or translocations in next-generation sequencing (NGS) reads are used. The presence of mutations or genetic alterations in the NF1 gene can be assessed in a sample, e.g., a biopsy sample obtained from the subject.

The domain architecture of neurofibromin is complex and comprises, from N-terminal to C-terminal ends:
(1) an N-heat domain comprising, among others, the cysteine and serine-rich domain/GTPase activating domain (CSRD) and the tubulin binding domain (TBD);
(2) a GTPase-activating domain (GAP) related domain (GRD), which promotes the hydrolysis of active Ras-GTP to the inactive form of Ras-GDP;
(3) a Sec14 homologous segment;
(4) a Pleckstrin homology (PH)-like domain; and
(5) a C-heat domain comprising a Heat-like repeat domain (HLR) and a C-terminal domain (CTD) where the Syndecan-binding domain (SBD) is found.

Additionally, the Sec14, PH-like and HLR domains form part of the so-called leucine-rich domain (LRD). Dimerization sites are found interspersed within the N-heat domain (at the far N-terminal end and at the TBD) and especially within the C-heat domain.

In principle, the NF1-mutant tumor to be treated in accordance with the invention may have one or more mutations or genetic alterations (particularly one or more inactivating mutations or inactivating genetic alterations) in the NF1 gene sequence for any one (or several ones) of the above-mentioned domains or segments of the NF1 gene product neurofibromin.

Thus, for example, the NF1-mutant tumor may be an NF1-mutant tumor (e.g., an NF1-mutant AML) having one or more inactivating mutations located in the GTPase activating domain (GAD) related domain (GRD) of NF1.

In some embodiments, the NF1-mutant tumor has one or more inactivating mutations located in the cysteine and serine-rich domain/GTPase activating domain (CSRD) of NF1.

In some embodiments, the NF1-mutant tumor has one or more inactivating mutations located in the leucin-rich domain (LRD) of NF1.

In some embodiments, the NF1-mutant tumor has one or more inactivating mutations located in at least one dimerization interface of NF1.

Furthermore, the present invention specifically relates to the treatment of an NF1-mutant tumor in a subject which has been determined (or diagnosed) to have an NF1-mutant tumor (e.g., any of the above-described specific or exemplary NF1-mutant tumors). In particular, the invention also relates to a corresponding treatment (or a corresponding method or use) which encompasses a step of testing a subject having a tumor for the presence of one or more mutations or genetic alterations affecting the NF1 gene (including, e.g., any of the above-described mutations or genetic alterations) and, if such mutations or genetic alterations have been determined to be present, a subsequent step of administering an LSD1 inhibitor to the subject.

Accordingly, the invention provides an LSD1 inhibitor for use in the treatment of an NF1-mutant tumor in a subject which has been determined (or diagnosed) to have an NF1-mutant tumor. The invention also relates to a pharmaceutical composition comprising an LSD1 inhibitor and optionally one or more pharmaceutically acceptable excipients for use in the treatment of an NF1-mutant tumor in a subject which has been determined (or diagnosed) to have an NF1-mutant tumor. The invention likewise provides a method of treating an NF1-mutant tumor in a subject which has been determined (or diagnosed) to have an NF1-mutant tumor, comprising administering a therapeutically effective amount of an LSD1 inhibitor (or a therapeutically effective amount of a pharmaceutical composition comprising an LSD1 inhibitor and optionally one or more pharmaceutically acceptable excipients) to the subject in need thereof. Moreover, the invention relates to the use of an LSD1 inhibitor for the treatment of an NF1-mutant tumor in a subject which has been determined (or diagnosed) to have an NF1-mutant tumor. The invention further relates to the use of an LSD1 inhibitor for the preparation of a medicament (or a pharmaceutical composition) for the treatment of an NF1-mutant tumor in a subject which has been determined (or diagnosed) to have an NF1-mutant tumor.

Moreover, the invention provides an LSD1 inhibitor for use in the treatment of an NF1-mutant tumor, wherein said use comprises a step of testing a subject having a tumor for the presence of one or more mutations or genetic alterations affecting the NF1 gene (including, e.g., any of the above-mentioned mutations or genetic alterations) and, if said mutations or genetic alterations affecting the NF1 gene have been determined to be present, a step of administering the LSD1 inhibitor to the subject. The invention also relates to a pharmaceutical composition comprising an LSD1 inhibitor and optionally one or more pharmaceutically acceptable excipients for use in the treatment of an NF1-mutant tumor, wherein said use comprises a step of testing a subject having a tumor for the presence of one or more mutations or genetic alterations affecting the NF1 gene (including, e.g., any of the above-mentioned mutations or genetic alterations) and, if said mutations or genetic alterations affecting the NF1 gene have been determined to be present, a step of administering the pharmaceutical composition to the subject. The invention likewise provides a method of treating an NF1-mutant tumor in a subject in need thereof, comprising a step of testing a subject having a tumor for the presence of one or more mutations or genetic alterations affecting the NF1 gene (including, e.g., any of the above-mentioned mutations or genetic alterations) and, if said mutations or genetic alterations affecting the NF1 gene have been determined to be present, a step of administering a therapeutically effective amount of an LSD1 inhibitor (or a therapeutically effective amount of a pharmaceutical composition comprising an LSD1 inhibitor and optionally one or more pharmaceutically acceptable excipients) to the subject. Moreover, the invention relates to the use of an LSD1 inhibitor for the treatment of an NF1-mutant tumor, wherein said treatment comprises testing a subject having a tumor for the presence of one or more mutations or genetic alterations affecting the NF1 gene (including, e.g., any of the above-mentioned mutations or genetic alterations) and, if said mutations or genetic alterations affecting the NF1 gene have been determined to be present, administering the LSD1 inhibitor to the subject. The invention further relates to the use of an LSD1 inhibitor for the preparation of a medicament (or a pharmaceutical composition) for the treatment of an NF1-mutant tumor, wherein said treatment comprises testing a subject having a tumor for the presence of one or more mutations or genetic alterations affecting the NF1 gene (including, e.g., any of the above-mentioned mutations or genetic alterations) and, if said mutations or genetic alterations affecting the NF1 gene have been determined to be present, administering the medicament (or the pharmaceutical composition) to the subject.

While mutations or genetic alternations in the NF1 gene are a hallmark of neurofibromatosis type I, a hereditary disease, such mutations or genetic alternations contribute to the development of a range of different tumors both in subjects with neurofibromatosis type I and in subjects without neurofibromatosis type I. Thus, in some embodiments, the present invention relates to the treatment of an NF1-mutant tumor in a subject having neurofibromatosis type I. Accordingly, the invention relates to an LSD1 inhibitor (or a pharmaceutical composition comprising an LSD1 inhibitor) for use in the treatment of an NF1-mutant tumor, wherein the LSD1 inhibitor (or the pharmaceutical composition) is administered to a subject having neurofibromatosis type I. Yet, the present invention also specifically relates to the treatment of an NF1-mutant tumor in a subject not having neurofibromatosis type I. Accordingly, in some embodiments, the invention relates to an LSD1 inhibitor (or a pharmaceutical composition comprising an LSD1 inhibitor) for use in the treatment of an NF1-mutant tumor, wherein the LSD1 inhibitor (or the pharmaceutical composition) is administered to a subject not having neurofibromatosis type I.

The NF1-mutant tumor to be treated may further be a metastatic NF1-mutant malignant tumor, i.e. a metastatic NF1-mutant cancer. Accordingly, the NF1-mutant tumor to be treated may be a primary NF1-mutant cancer that has formed metastases, i.e., that has spread to one or more other parts of the body of the subject.

The NF1-mutant tumor to be treated may also be a relapsed or refractory NF1-mutant cancer.

The therapeutic effects of LSD1 inhibitors in the treatment of NF1-mutant tumors can be further confirmed in additional in vitro or in vivo experiments, as well as in clinical trials in humans, which can be readily set up by those skilled in the art of drug development.

### LSD1 Inhibitors

As used herein, an “LSD1 inhibitor” means a compound/substance that reduces, decreases, blocks or inhibits the gene expression, activity or function of LSD1. Compounds which act as inhibitors of LSD1 are known in the art. Any molecule acting as an LSD1 inhibitor can, in principle, be used in the context of the present invention. Preferably, the LSD1 inhibitor is a small molecule. Moreover, the LSD1 inhibitor may be an irreversible LSD1 inhibitor or a reversible LSD1 inhibitor. As also demonstrated in the examples section below, both irreversible and reversible LSD1 inhibitors can be used for the treatment of NF1-mutant tumors in accordance with the present invention. Prototypical irreversible LSD1 inhibitors include cyclopropylamine-based compounds like iadademstat and bomedemstat, which are among the LSD1 inhibitors used in the examples section. A representative example of a reversible LSD1 inhibitor is the compound pulrodemstat, which has also been used in the examples section. Preferably, the LSD1 inhibitor is a selective LSD1 inhibitor; as used herein, a “selective LSD1 inhibitor” means an LSD1 inhibitor which exhibits a selectivity of at least 10-fold (preferably at least 100-fold) for LSD1 over other FAD-dependent monoamine oxidases, particularly over MAO-A and MAO-B (which can be assessed, e.g., by determining IC₅₀ values for LSD1, MAO-A and MAO-B).

An exemplary list of small-molecule LSD1 inhibitors is provided in the table below:

| | | International |
|---|---|---|
| | Generic | Non-proprietary |
| Chemical name | name/Code | Name (INN) |
| (trans)-N1-((1R,2S)-2-phenylcyclopropyl)cyclohexane-1,4-diamine | ORY-1001 | iadademstat |
| 4-[2-(4-aminopiperidin-1-yl)-5-(3-fluoro-4-methoxyphenyl)-1-methyl-6-oxo- | CC-90011 | pulrodemstat |
| 1,6-dihydropyrimidin-4-yl]-2-fluorobenzonitrile | | |
| N-[(2S)-5-{[(1R,2S)-2-(4-fluorophenyl)cyclopropyl]amino}-1-(4- | IMG-7289 | bomedemstat |
| methylpiperazin-1-yl)-1-oxopentan-2-yl]-4-(1H-1,2,3-triazol-1-yl)benzamide | | |
| (E)-N′-(1-(5-chloro-2-hydroxyphenyl)ethylidene)-3-((4-methylpiperazin-1- | SP-2577 | seclidemstat |
| yl)sulfonyl)benzohydrazide | | |
| 1-((4-(methoxymethyl)-4-(((1R,2S)-2- | | |
| phenylcyclopropylamino)methyl)piperidin-1- | | |
| yl)methyl)cyclobutanecarboxylic acid | | |
| 3-(cyanomethyl)-3-(4-{[(1R,2S)-2-phenylcyclopropyl]amino}piperidin-1- | | |
| yl)azetidine-1-sulfonamide | | |
| 5-((((1R,2S)-2-(4-(benzyloxy)phenyl)cyclopropyl)amino)methyl)-1,3,4- | ORY-2001 | vafidemstat |
| oxadiazol-2-amine | | |
| N-((trans)-2-phenylcyclopropyl)piperidin-4-amine | OG-668, also | |
| | known as | |
| | GSK-LSD1 | |
| N-[4-[(trans)-2-[(cyclopropylmethyl)amino]cyclopropyl]phenyl]-1-methyl-1H- | T-3775440 | |
| pyrazole-4-carboxamide | | |
| 1-morpholino-3-(4-(((1R,2S)-2-phenylcyclopropyl)amino)piperidin-1- | MRTX1519 | |
| yl)propan-1-one | | |
| 4-[[4-[[[(1R,2S)-2-phenylcyclopropyl]amino]methyl]-1-piperidinyl]methyl]- | GSK2879552 | |
| benzoic acid | | |
| 4-[5-[(3S)-3-aminopyrrolidine-1-carbonyl]-2-[2-fluoro-4-(2-hydroxy-2- | | |
| methyl-propyl)phenyl]phenyl]-2-fluoro-benzonitrile | | |
| 2-(6-(((1R,2S)-2-((E)-1-phenylbut-1-en-2-yl)cyclopropyl)amino)-2- | | |
| azaspiro[3.3]heptan-2-yl)ethanol | | |
| 2-(6-(((1R,2S)-2-((E)-1-phenylbut-1-en-2-yl)cyclopropyl)amino)-2- | | |
| azaspiro[3.3]heptan-2-yl)propane-1,3-diol | | |
| 4-[5-[(3S)-3-aminopyrrolidine-1-carbonyl]-2-[2-fluoro-4-(2-hydroxy-2- | TAS1440 | |
| methyl-propyl)phenyl]phenyl]-2-fluoro-benzonitrile | | |

The LSD1 inhibitor to be used in accordance with the present invention may thus be, e.g., any one of the specific compounds listed in the table above, or a pharmaceutically acceptable salt of any one of these compounds.

In some embodiments, the LSD1 inhibitor is an LSD1 inhibitor known in the art, including, e.g., any one of the compounds disclosed in WO2010/043721, WO2010/084160, WO2010/143582, WO2011/035941, WO2011/042217, WO2011/131576, WO2011/131697, WO2012/013727, WO2012/013728, WO2012/045883, WO2012/135113, WO2013/022047, EP2743256A1, WO2013/025805, WO2013/057320, WO2013/057322, WO2014/058071, EP2907802A1, WO2014/084298, EP2927212A1, WO2014/086790, WO2014/164867, WO2014/194280, WO2014/205213, WO2015/021128, WO2015/031564, WO2015/089192, WO2015/120281, WO2015/123408, WO2015/123424, WO2015/123437, WO2015/123465, WO2015/134973, WO2015/168466, WO2015/181380, WO2015/200843, WO2016/003917, WO2016/004105, WO2016/007722, WO2016/007727, WO2016/007731, WO2016/007736, WO2016/034946, WO2016/037005, WO2016/123387, WO2016/130952, WO2016/161282, WO2016/172496, WO2016/177656, WO2017/004519, WO2017/027678, WO2017/079476, WO2017/079670, WO2017/090756, EP3381896A1, WO2017/109061, WO2017/116558, WO2017/149463, WO2017/157322, EP3431471A1, WO2017/184934, WO2017/195216, WO2017/198780, WO2017/215464, EP3486244A1, WO2018/081342, WO2018/081343, WO2018/137644, EP3575285A1, WO2018/213211, WO2018/216800, EP3632897A1, WO2018/226053, WO2018/234978, WO2019/009412, WO2019/034774, WO2019/054766, WO2019/217972, WO2019/222069, WO2020/015745, EP3825309A1, WO2020/047198, WO2020/052647, WO2020/052649, EP3851440A1, WO2020/138398, WO2020/159285, EP3907225A1, WO2021/058024, WO2021/095835, WO2021/175079, WO2022/072811, WO2022/171044, WO2022/188709, WO2022/240886, WO2022/267495, WO2023/069884, WO2023/284651, US2017-0283397, US2022-0064126, CN103054869, CN103319466, CN104119280, CN105541806, CN105924362, CN105985265, CN106045862, CN106045881, CN106432248, CN106478639, CN106831489, CN106928235, CN107033148 CN107174584, CN107176927, CN107459476, CN107474011, CN107501169, CN107936022, CN108530302, CN109265462, CN109293664, CN109535019, CN110204551, CN110478352, CN111072610, CN111454252, CN112110936, CN112409310, CN112920130, CN113087712, CN113105479, CN113264903, CN113582906, CN113599380, CN114502561, CN114805205, CN114805261, KR20190040763, or KR20190040783, each of which is incorporated herein by reference in its entirety (including, in particular, the compounds described in the examples section of each one of these documents). Accordingly, the LSD1 inhibitor may be, e.g., a compound disclosed in any one of the aforementioned documents (including, e.g., in the examples section of any one of these documents), wherein said compound may be used in non-salt form or in the form of a pharmaceutically acceptable salt.

In some embodiments, the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl) piperidin-1-yl)methyl)cyclobutanecarboxylic acid, 3-(cyanomethyl)-3-(4-{[(1R,2S)-2-phenylcyclopropyl]amino}piperidin-1-yl) azetidine-1-sulfonamide, vafidemstat, 4-[5-[(3S)-3-aminopyrrolidine-1-carbonyl]-2-[2-fluoro-4-(2-hydroxy-2-methyl-propyl)phenyl]phenyl]-2-fluoro-benzonitrile, and pharmaceutically acceptable salts thereof (i.e., pharmaceutically acceptable salts of any one of the aforementioned compounds).

Iadademstat is a selective and irreversible LSD1 inhibitor. Iadademstat is the INN for the compound of formula:

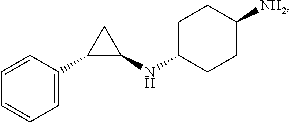 [SMILES: N[C@H]1CC[C@H](N[C@@H]2C[C@H]2c2ccccc2)CC1]

[CAS Reg. No. 1431304-21-0], which is also known as ORY-1001 or (trans)-N1-((1R,2S)-2-phenylcyclopropyl)cyclohexane-1,4-diamine. Iadademstat has been described, e.g., in Example 5 of WO2013/057322. Pharmaceutically acceptable salts of iadademstat, including hydrochloride salts (particularly iadademstat dihydrochloride), are also described in WO2013/057322.

Pulrodemstat is a reversible LSD1 inhibitor of formula

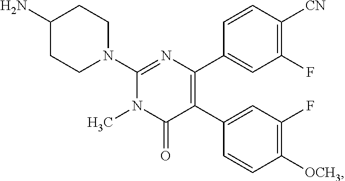 [SMILES: COc1ccc(-c2c(-c3ccc(C#N)c(F)c3)nc(N3CCC(N)CC3)n(C)c2=O)cc1F]

[CAS Reg. No. 1821307-10-1], also known as CC-90011, with the chemical name 4-[2-(4-aminopiperidin-1-yl)-5-(3-fluoro-4-methoxyphenyl)-1-methyl-6-oxo-1,6-dihydropyrimidin-4-yl]-2-fluorobenzonitrile. Pulrodemstat has been described, e.g., in WO2015/168466 and WO2017/79670. Pharmaceutically acceptable salts thereof are also described therein, including a besylate salt.

Bomedemstat is an irreversible LSD1 inhibitor of formula

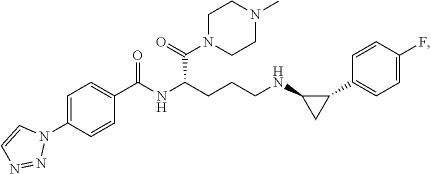 [SMILES: CN1CCN(C(=O)[C@H](CCCN[C@@H]2C[C@H]2c2ccc(F)cc2)NC(=O)c2ccc(-n3ccnn3)cc2)CC1]

[CAS Reg. No. 1990504-34-1], also known as IMG-7289, with the chemical name N-[(2S)-5-{[(1R,2S)-2-(4-fluorophenyl)cyclopropyl]amino}-1-(4-methylpiperazin-1-yl)-1-oxopentan-2-yl]-4-(1H-1,2,3-triazol-1-yl)benzamide. Bomedemstat has been described, e.g., in WO2016/130952 and WO2018/35259. Pharmaceutically acceptable salts thereof are also described therein, including a bis-tosylate salt.

Seclidemstat is an LSD1 inhibitor of formula

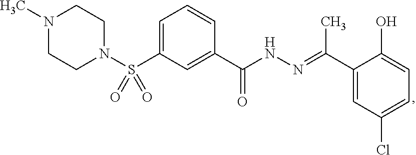 [SMILES: C/C(=N\NC(=O)c1cccc(S(=O)(=O)N2CCN(C)CC2)c1)c1cc(Cl)ccc1O ; ","]

[CAS Reg. No. 1423715-37-0], also known as SP-2577, with the chemical name (E)-N′-(1-(5-chloro-2-hydroxyphenyl)ethylidene)-3-((4-methylpiperazin-1-yl) sulfonyl)benzohydrazide. Seclidemstat has been described, e.g., in WO2013/025805 and WO2014/205213.

1-((4-(Methoxymethyl)-4-(((1R,2S)-2-phenylcyclopropylamino)methyl) piperidin-1-yl)methyl)cyclobutanecarboxylic acid is an irreversible LSD1 inhibitor which is described, e.g., in WO2015/123465 and WO2017/27678. Pharmaceutically acceptable salts thereof are also described therein, including a para-toluenesulfonate salt. The structure of this compound can be depicted as follows:

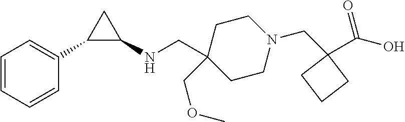 [SMILES: COCC1(CN[C@@H]2C[C@H]2c2ccccc2)CCN(CC2(C(=O)O)CCC2)CC1]

3-(Cyanomethyl)-3-(4-{[(1R,2S)-2-phenylcyclopropyl]amino}piperidin-1-yl) azetidine-1-sulfonamide is an irreversible LSD1 inhibitor which is described, e.g., in WO2020/047198. Pharmaceutically acceptable salts thereof are also described therein. The structure of this compound can be depicted as follows:

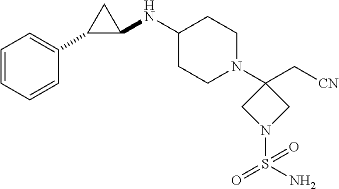

Vafidemstat is an irreversible LSD1 inhibitor of formula:

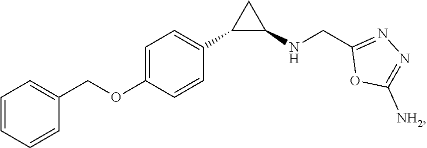 [SMILES: Nc1nnc(CN[C@@H]2C[C@H]2c2ccc(OCc3ccccc3)cc2)o1]

which is also known as ORY-2001, 5-((((1R,2S)-2-(4-(benzyloxy)phenyl)cyclopropyl)amino)methyl)-1,3,4-oxadiazol-2-amine, or (−) 5-((((trans)-2-(4-(benzyloxy)phenyl)cyclopropyl)amino)methyl)-1,3,4-oxadiazol-2-amine. Vafidemstat has been described, e.g., in Example 35 of WO2012/13728.

4-[5-[(3S)-3-Aminopyrrolidine-1-carbonyl]-2-[2-fluoro-4-(2-hydroxy-2-methyl-propyl)phenyl]phenyl]-2-fluoro-benzonitrile is an LSD1 inhibitor which is described, e.g., in WO2017/090756 (or EP3381896A1; see Example 37), WO2021/095835, WO2022/240886, and WO2023/054547. Also described therein are pharmaceutically acceptable salts of this compound, including a benzoic acid salt (or benzoate salt), a sorbic acid salt, a succinic acid salt, an L-tartaric acid salt, a hydrochloric acid salt, a hemi-fumarate salt, a mono-fumarate salt, a hemi-oxalate salt, a mono-oxalate salt, a mesylate salt, an esylate salt, or a maleate salt. Specific solid forms of this compound are described in WO2022/240886. This compound is also referred to herein as “TAS1440”. The structure of this compound can be depicted as follows:

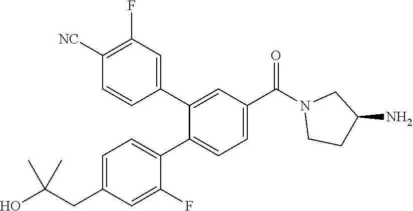 [SMILES: CC(C)(O)Cc1ccc(-c2ccc(C(=O)N3CC[C@H](N)C3)cc2-c2ccc(C#N)c(F)c2)c(F)c1]

Further examples of the LSD1 inhibitor include SYHA1807 or a pharmaceutically acceptable salt thereof, or JBI-802 or a pharmaceutically acceptable salt thereof.

In some embodiments, the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl) piperidin-1-yl)methyl)cyclobutanecarboxylic acid, 3-(cyanomethyl)-3-(4-{[(1R,2S)-2-phenylcyclopropyl]amino}piperidin-1-yl) azetidine-1-sulfonamide, 4-[5-[(3S)-3-aminopyrrolidine-1-carbonyl]-2-[2-fluoro-4-(2-hydroxy-2-methyl-propyl)phenyl]phenyl]-2-fluoro-benzonitrile, and pharmaceutically acceptable salts thereof.

In particular, the LSD1 inhibitor may be selected from the group consisting of iadademstat, pulrodemstat, bomedemstat, seclidemstat, 1-((4-(methoxymethyl)-4-(((1R,2S)-2-phenylcyclopropylamino)methyl) piperidin-1-yl)methyl)cyclobutanecarboxylic acid, 3-(cyanomethyl)-3-(4-{[(1R,2S)-2-phenylcyclopropyl]amino}piperidin-1-yl) azetidine-1-sulfonamide, and pharmaceutically acceptable salts thereof.

In preferred embodiments, the LSD1 inhibitor is selected from the group consisting of iadademstat, pulrodemstat, bomedemstat, and pharmaceutically acceptable salts thereof. In some embodiments, the LSD1 inhibitor is pulrodemstat or a pharmaceutically acceptable salt thereof (e.g., pulrodemstat besylate). In some embodiments, the LSD1 inhibitor is bomedemstat, or a pharmaceutically acceptable salt thereof (e.g., bomedemstat bis-tosylate).

A particularly preferred LSD1 inhibitor is iadademstat or a pharmaceutically acceptable salt thereof. In some embodiments, iadademstat is used as the dihydrochloride salt (i.e., iadademstat dihydrochloride).

Unless specifically indicated otherwise, any reference to an LSD1 inhibitor (e.g., iadademstat) throughout the present description and claims includes such LSD1 inhibitor in non-salt form and any of its pharmaceutically acceptable salts. When the LSD1 inhibitor is iadademstat, it is preferably used in the form of a pharmaceutically acceptable salt, preferably a hydrochloride salt, more preferably the dihydrochloride salt.

### Pharmaceutical Formulations

The LSD1 inhibitor to be used in accordance with the present invention, as well as any pharmaceutical composition comprising an LSD1 inhibitor to be used in accordance with the invention, may be administered by any route appropriate to the condition to be treated. Exemplary routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Preferably, the LSD1 inhibitor (or the corresponding pharmaceutical composition) is administered orally. The LSD1 inhibitor to be used in accordance with the present invention may be administered in any convenient pharmaceutical composition or formulation, e.g., as tablets, powders, capsules, solutions, dispersions, suspensions, syrups, sprays, suppositories, gels, emulsions, patches, etc. Such compositions/formulations may comprise components conventional in pharmaceutical preparations, e.g., diluents, carriers, pH modifiers, preservatives, solubilizers, stabilizers, wetting agents, emulsifiers, sweeteners, colorants, flavorants, salts for varying the osmotic pressure, buffers, masking agents, antioxidants, and/or further active agents. They can also comprise still other therapeutically active or therapeutically valuable substances.

A typical formulation is prepared by mixing an LSD1 inhibitor and one or more pharmaceutically acceptable excipients. Suitable excipients are well known to those skilled in the art and are described in detail in, e.g., “Ansel's Pharmaceutical Dosage Forms and Drug Delivery Systems” (2004) Lippincott, Williams & Wilkins, Philadelphia; “Remington: The Science and Practice of Pharmacy” (2000) Lippincott, Williams & Wilkins, Philadelphia; or “Handbook of Pharmaceutical Excipients” (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and/or other known additives to provide an elegant presentation of the active agent or aid in the manufacturing of the pharmaceutical product (i.e., medicament).

For oral delivery, the LSD1 inhibitor can be incorporated into a formulation that includes pharmaceutically acceptable carriers such as binders (e.g., gelatin, cellulose, or gum tragacanth), excipients (e.g., starch or lactose), lubricants (e.g., magnesium stearate or silicon dioxide), disintegrating agents (e.g., alginate, Primogel, or corn starch), and sweetening or flavoring agents (e.g., glucose, sucrose, saccharin, methyl salicylate, or peppermint). The formulation can be orally delivered, e.g., in the form of enclosed gelatin capsules or compressed tablets. Capsules and tablets can be prepared by any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of a suspension, syrup, chewing gum, wafer, elixir, and the like. If desired, conventional agents for modifying flavors, tastes, colors, and shapes of the special forms can also be included. In addition, for convenient administration by enteral feeding tube in subjects unable to swallow, the active compounds can be dissolved in an acceptable lipophilic vegetable oil vehicle, such as olive oil, corn oil or safflower oil.

The LSD1 inhibitor can also be administered parenterally in the form of a solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetates, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Subcutaneous implantation for sustained release of the LSD1 inhibitor may also be a suitable route of administration. This entails surgical procedures for implanting an LSD1 inhibitor in any suitable formulation into a subcutaneous space, e.g., beneath the anterior abdominal wall. See, e.g., Wilson et al. (1984) J. Clin. Psych. 45:242-247. Hydrogels can be used as a carrier for the sustained release of LSD1 inhibitors. Hydrogels are generally known in the art. They are typically made by crosslinking high molecular weight biocompatible polymers into a network, which swells in water to form a gel-like material. Preferably, hydrogels are biodegradable or biosorbable. For the purpose of this invention, hydrogels made of polyethylene glycols, collagen, or poly(glycolic-co-L-lactic acid) may be useful; see, e.g., Phillips et al. (1984) J. Pharmaceut. Sci., 73:1718-1720.

The pharmaceutical compositions, like oral and parenteral compositions, can be formulated in unit dosage forms for ease of administration and uniformity of dosage. As used herein, “unit dosage forms” refers to physically discrete units suitable as unitary dosages for administration to subjects, each unit containing a predetermined quantity of active ingredient calculated to produce the desired therapeutic effect, in association with one or more suitable pharmaceutical carriers.

Suitable oral dosage forms for iadademstat are disclosed, for example, in WO2019/211491.

In particular, iadademstat may be provided in the form of a solid oral dosage form, such as, e.g., tablets or capsules. Alternatively, iadademstat may also be provided in the form of an oral liquid composition, particularly an oral solution, such as, e.g., an oral aqueous solution (a corresponding oral solution, including an oral aqueous solution, may be prepared, e.g., from a powder for reconstitution). As explained above, it is preferred that iadademstat is used in the form of iadademstat dihydrochloride.

For the treatment of an NF1-mutant tumor, the LSD1 inhibitor (or the corresponding pharmaceutical composition) can be administered in any appropriate manner, as determined by a person skilled in the medical arts. An appropriate dose and suitable duration and frequency of administration can vary within wide limits and will be determined by such factors as the condition of the subject, the specific type and severity of the disease, the particular form of the active ingredient(s), and the method of administration, among others. In general, an appropriate dose and administration regimen provides the LSD1 inhibitor in an amount sufficient to provide therapeutic benefit, for example an improved clinical outcome, such as more frequent complete or partial remissions, or longer disease-free and/or overall survival, or lessening of symptoms severity, or any other objectively identifiable improvement as noted by the clinician. Therapeutically effective doses may generally be assessed or extrapolated using experimental models like dose-response curves derived from in vitro or animal model test systems, or from clinical trials in humans.

Suitable doses and dosing regimens for the LSD1 inhibitor will be dependent on the specific LSD1 inhibitor used, its LSD1 inhibitory potency, its pharmacokinetic profile and other factors, as well known by those skilled in the art. Iadademstat is a highly potent active pharmaceutical ingredient (HPAPI). The anticipated daily dose is thus very low, e.g., lower than 1 mg per day. Accordingly, the drug load in a pharmaceutical formulation (including, e.g., a solid oral form) will typically also be very low (e.g., less than 1 mg of API per 100 mg of solid oral form). In general, in the case of oral administration (e.g., as tablets, capsules, or as an oral solution, including e.g. an oral aqueous solution) to an adult human subject (i.e., a human subject having an age of 18 years or more), a daily dosage of about 50 μg to about 300 μg, preferably of about 75 μg to about 300 μg (e.g., about 75 μg, about 100 μg, about 125 μg, about 150 μg, about 175 μg, about 200 μg, about 225 μg, about 250 μg, about 275 μg, or about 300 μg, or any range between any two of the aforementioned daily dosages), of iadademstat as described herein should be appropriate, although these limits may be adjusted when necessary. For example, the aforementioned dosages may be lowered for paediatric use, particularly for the oral administration to a human subject having less than 18 years of age (e.g., having 0 to 2 years, 2 to 12 years, or 12 to less than 18 years). The term “μg” (or “μg”), as used herein, refers to micrograms.

In some embodiments, the LSD1 inhibitor is iadademstat (or a pharmaceutically acceptable salt thereof, e.g., iadademstat dihydrochloride) and is administered five days on/two days off (5/2) per week.

In some embodiments, the LSD1 inhibitor is iadademstat (or a pharmaceutically acceptable salt thereof, e.g., iadademstat dihydrochloride) and is administered orally to an adult human subject at a daily dose of about 50 μg to about 300 μg, preferably of about 75 μg to about 300 μg (e.g., about 100 μg to about 300 μg), five days on/two days off (5/2) per week. Doses as reflected herein for iadademstat relate to the corresponding amount of the iadademstat free base. In some embodiments, iadademstat is administered orally at a daily dose of about 75 μg five days on/two days off (5/2) per week. In some embodiments, iadademstat is administered orally at a daily dose of about 100 μg five days on/two days off (5/2) per week. In some embodiments, iadademstat is administered orally at a daily dose of about 150 μg five days on/two days off (5/2) per week. In some embodiments, iadademstat is administered orally at a daily dose of about 200 μg five days on/two days off (5/2) per week. In some embodiments, iadademstat is administered orally at a daily dose of about 250 μg five days on/two days off (5/2) per week. In some embodiments, iadademstat is administered orally at a daily dose of about 300 μg five days on/two days off (5/2) per week. As explained above, these dosages may be lowered for paediatric use.

### Combination Treatments

The LSD1 inhibitor to be used in accordance with the present invention can be administered in monotherapy (e.g., without concomitantly administering any further therapeutic agents, or without concomitantly administering any further anticancer agents). Accordingly, the invention relates to an LSD1 inhibitor (or a pharmaceutical composition comprising an LSD1 inhibitor and optionally one or more pharmaceutically acceptable excipients) for use in the monotherapeutic treatment of an NF1-mutant tumor (preferably an NF1-mutant cancer). The invention likewise relates to corresponding methods and uses for the monotherapeutic treatment of an NF1-mutant tumor (preferably an NF1-mutant cancer).

However, the LSD1 inhibitor can also be administered in combination with one or more further therapeutic agents, particularly one or more further anticancer agents. If the LSD1 inhibitor is used in combination with a further anticancer agent, the dose of each compound may differ from that when the corresponding compound is used alone, in particular, a lower dose of either one or both compounds may be used.

The combination of the LSD1 inhibitor with one or more further therapeutic agents (e.g., one or more further anticancer agents) may comprise the simultaneous/concomitant administration of the LSD1 inhibitor and the further therapeutic agent(s), either in a single pharmaceutical formulation or in separate pharmaceutical formulations, or the sequential/separate administration of the LSD1 inhibitor and the further therapeutic agent(s). If administration is sequential, either the LSD1 inhibitor or the one or more further therapeutic agents may be administered first. If administration is simultaneous, the one or more further therapeutic agents may be included in the same pharmaceutical formulation as the LSD1 inhibitor, or they may be administered in two or more distinct/separate pharmaceutical formulations. It will be appreciated that administering the LSD1 inhibitor and the further therapeutic agent(s) in separate pharmaceutical formulations is expedient, e.g., if the respective agents are administered by different routes and/or using different administration schedules/regimens.

Moreover, the LSD1 inhibitor can also be administered in combination with physical therapy, particularly radiotherapy. The invention likewise relates to the combined use of an LSD1 inhibitor with one or more further therapeutic agents (particularly one or more further anticancer agents) and with physical therapy (particularly radiotherapy). Physical therapy (or radiotherapy) may commence before, after, or simultaneously with the administration of the LSD1 inhibitor (e.g., about 1 to 72 hours before or after the administration of the LSD1 inhibitor).

Accordingly, the invention relates to an LSD1 inhibitor for use in the treatment of an NF1-mutant tumor (preferably an NF1-mutant cancer) in combination with one or more further therapeutic agents (particularly one or more further anticancer agents) and/or in combination with radiotherapy. The invention also relates to a pharmaceutical composition comprising an LSD1 inhibitor and optionally one or more pharmaceutically acceptable excipients for use in the treatment of an NF1-mutant tumor (preferably an NF1-mutant cancer) in combination with one or more further therapeutic agents (particularly one or more further anticancer agents) and/or in combination with radiotherapy. The invention further relates to an LSD1 inhibitor (or a pharmaceutical composition comprising an LSD1 inhibitor and optionally one or more pharmaceutically acceptable excipients) for use in the treatment of an NF1-mutant tumor (preferably an NF1-mutant cancer), wherein the LSD1 inhibitor (or the pharmaceutical composition comprising the LSD1 inhibitor) is administered in combination with one or more further therapeutic agents (particularly one or more further anticancer agents) and/or in combination with radiotherapy. The invention further relates to an LSD1 inhibitor (or a pharmaceutical composition comprising an LSD1 inhibitor and optionally one or more pharmaceutically acceptable excipients) for use in the treatment of an NF1-mutant tumor (preferably an NF1-mutant cancer), wherein the LSD1 inhibitor (or the pharmaceutical composition comprising the LSD1 inhibitor) is for use in combination with one or more further therapeutic agents (particularly one or more further anticancer agents) and/or in combination with radiotherapy. The invention further relates to: (i) an anticancer agent for use in the treatment of an NF1-mutant tumor (preferably an NF1-mutant cancer) in combination with an LSD1 inhibitor; (ii) an anticancer agent for use in the treatment of an NF1-mutant tumor (preferably an NF1-mutant cancer), wherein the anticancer agent is administered in combination with an LSD1 inhibitor; or (iii) an anticancer agent for use in the treatment of an NF1-mutant tumor (preferably an NF1-mutant cancer), wherein the anticancer agent is for use in combination with an LSD1 inhibitor.

The invention likewise provides a method of treating an NF1-mutant tumor (preferably an NF1-mutant cancer) in a subject in need thereof, comprising administering to the subject a therapeutically effective amount of an LSD1 inhibitor (or a therapeutically effective amount of a pharmaceutical composition comprising an LSD1 inhibitor and optionally one or more pharmaceutically acceptable excipients) in combination with a therapeutically effective amount of one or more further therapeutic agents (particularly one or more further anticancer agents) and/or in combination with radiotherapy. Moreover, the invention relates to the use of an LSD1 inhibitor for the treatment of an NF1-mutant tumor (preferably an NF1-mutant cancer) in combination with one or more further therapeutic agents (particularly one or more further anticancer agents) and/or in combination with radiotherapy. The invention also relates to the use of an anticancer agent for the treatment of an NF1-mutant tumor (preferably an NF1-mutant cancer) in combination with an LSD1 inhibitor. The invention furthermore relates to the use of an LSD1 inhibitor for the preparation of a medicament (or a pharmaceutical composition) for the treatment of an NF1-mutant tumor (preferably an NF1-mutant cancer) in combination with one or more further therapeutic agents (particularly one or more further anticancer agents) and/or in combination with radiotherapy. The invention also relates to the use of an anticancer agent for the preparation of a medicament for the treatment of an NF1-mutant tumor (preferably an NF1-mutant cancer) in combination with an LSD1 inhibitor. The invention likewise relates to the use of an LSD1 inhibitor and one or more further anticancer agents for the preparation of a medicament for the treatment of an NF1-mutant tumor (preferably an NF1-mutant cancer), wherein the medicament comprises the LSD1 inhibitor and the further anticancer agent(s) in the same pharmaceutical formulation or in separate pharmaceutical formulations. The invention also relates to the use of an LSD1 inhibitor for the preparation of a medicament for the treatment of an NF1-mutant tumor (preferably an NF1-mutant cancer), wherein said medicament is prepared for combined use (or for use in combination) with one or more further therapeutic agents (particularly one or more further anticancer agents) and/or with radiotherapy. The invention also relates to the use of an anticancer agent for the preparation of a medicament for the treatment of an NF1-mutant tumor (preferably an NF1-mutant cancer), wherein said medicament is prepared for combined use (or for use in combination) with an LSD1 inhibitor.

The present invention furthermore provides a combination product comprising, in the same pharmaceutical formulation or in separate pharmaceutical formulations, an LSD1 inhibitor and one or more further therapeutic agents (particularly one or more further anticancer agents), for use in the treatment of an NF1-mutant tumor (preferably an NF1-mutant cancer). The LSD1 inhibitor and the further therapeutic agent(s) (particularly the further anticancer agent(s) may thus be present in a single pharmaceutical formulation (i.e., in the same pharmaceutical formulation), or they may each be provided in a distinct (separate) pharmaceutical formulation.

The present invention also provides a pharmaceutical composition comprising an LSD1 inhibitor in combination with one or more further therapeutic agents (particularly one or more further anticancer agents), and one or more pharmaceutically acceptable excipients, for use in the treatment of an NF1-mutant tumor (preferably an NF1-mutant cancer).

The present invention further provides an article of manufacture (or a kit) comprising, in the same pharmaceutical formulation or in separate pharmaceutical formulations, an LSD1 inhibitor and one or more further therapeutic agents (particularly one or more further anticancer agents), for use in the treatment of an NF1-mutant tumor (preferably an NF1-mutant cancer).

The invention further provides a method of treating an NF1-mutant tumor (preferably an NF1-mutant cancer) in a subject in need thereof, comprising administering to the subject a therapeutically effective amount of the above-described combination product, the pharmaceutical composition or the article of manufacture. In particular, the invention provides a method of treating an NF1-mutant tumor (preferably an NF1-mutant cancer) in a subject in need thereof, comprising administering to the subject a therapeutically effective amount of a combination product comprising, in the same pharmaceutical formulation or in separate pharmaceutical formulations, an LSD1 inhibitor and one or more further therapeutic agents (particularly one or more further anticancer agents). The invention further provides a method of treating an NF1-mutant tumor (preferably an NF1-mutant cancer) in a subject in need thereof, comprising administering to the subject a therapeutically effective amount of an LSD1 inhibitor and a therapeutically effective amount of one or more further therapeutic agents (particularly one or more further anticancer agents).

The invention further provides the use of a combination comprising an LSD1 inhibitor and one or more further therapeutic agents (particularly one or more further anticancer agents) for the manufacture of a medicament (or a pharmaceutical composition) for the treatment of an NF1-mutant tumor (preferably an NF1-mutant cancer). The invention also provides the use of a combination comprising an LSD1 inhibitor and one or more further therapeutic agents (particularly one or more further anticancer agents) for the treatment of an NF1-mutant tumor (preferably an NF1-mutant cancer).

The anticancer agent(s) (particularly the “anticancer agent” or the “one or more further anticancer agents” as referred to in any of the paragraphs herein above) may be selected, for example, from a MEK inhibitor (particularly an inhibitor of MEK1 and/or MEK2; e.g., selumetinib), a Pi3K inhibitor (e.g., copanlisib), an mTOR inhibitor (e.g., temsirolimus), an ERK inhibitor (particularly an inhibitor of ERK1 and/or ERK2; e.g., ulixertinib), a kRAS inhibitor (e.g., sotorasib), an EGFR inhibitor (e.g., lapatinib), a cKIT inhibitor (e.g., imatinib), a proteasome inhibitor (e.g., bortezomib), a DNA intercalator (e.g., doxorubicin), a RAF inhibitor (particularly a BRAF inhibitor; e.g., sorafenib), a VEGFR inhibitor (e.g., cabozantinib), an ALK inhibitor (e.g., crizotinib), a glutaminase inhibitor (e.g., telaglenastat), a JAK inhibitor (or a Janus kinase inhibitor; e.g., tofacitinib), a PLK1 inhibitor (e.g., volasertib), a Bcl2 inhibitor (e.g., venetoclax), an HDAC inhibitor (e.g., vorinostat), an HSP90 inhibitor (e.g., onalespib), a Wnt/β-catenin pathway inhibitor (e.g., OMP-18R5), an Aurora kinase inhibitor (e.g., alisertib), an MDM2 inhibitor (e.g., alrizomadlin), a CDK4/6 inhibitor (e.g., abemaciclib), a YAP/TAZ pathway inhibitor (e.g., pazopanib), an SOS inhibitor (e.g., BI 1701963), a Grb2 inhibitor (e.g., BP1001), a BET inhibitor (including, in particular, a BRD4 inhibitor; e.g., GSK1210151A), an AKT inhibitor (e.g., ipatasertib), an MNK inhibitor (e.g., ETC-206), an NTRK inhibitor (e.g., entrectinib), an SPH2 inhibitor (e.g., JAB-3068), and a PP2A inhibitor (e.g., LB100).

The MEK inhibitor may be, e.g., selumetinib, trametinib, cobimetinib, binimetinib, mirdametinib, pimasertib, refametinib, zapnometinib, avutometinib, HL-085, FCN-159, TAK-733, or a pharmaceutically acceptable salt of any one of these agents. The Pi3K inhibitor may be, e.g., copanlisib, alpelisib, idelalisib, duvelisib, umbralisib, buparlisib, zandelisib, linperlisib, parsaclisib, leniolisib, paxalisib, inavolisib, serabelisib, pictilisib, taselisib, tenalisib, eganelisib, GSK2636771, MEN1611, AMG-319, or a pharmaceutically acceptable salt of any one of these agents. The mTOR inhibitor may be, e.g., temsirolimus, everolimus, sirolimus, or a pharmaceutically acceptable salt of any one of these agents. The ERK inhibitor may be, e.g., ulixertinib or a pharmaceutically acceptable salt thereof. The KRAS inhibitor may be, e.g., sotorasib, adagrasib, or a pharmaceutically acceptable salt of any one of these agents. The EGFR inhibitor may be, e.g., lapatinib, gefitinib, erlotinib, osimertinib, afatinib, or a pharmaceutically acceptable salt of any one of these agents. The cKIT inhibitor may be, e.g., imatinib, sorafenib, lapatinib, sunitinib, or a pharmaceutically acceptable salt of any one of these agents. The proteasome inhibitor may be, e.g., bortezomib, carfilzomib, ixazomib, or a pharmaceutically acceptable salt of any one of these agents. The DNA intercalator may be, e.g., doxorubicin, daunorubicin, epirubicin, idarubicin, or a pharmaceutically acceptable salt of any one of these agents. The RAF inhibitor may be, e.g., sorafenib, encorafenib, dabrafenib, vemurafenib, or a pharmaceutically acceptable salt of any one of these agents. The VEGFR inhibitor may be, e.g., cabozantinib, axatinib, lenvatinib, nintedanib, pazopanib, regorafenib, sorafenib, sunitinib, vandetanib, or a pharmaceutically acceptable salt of any one of these agents. The ALK inhibitor may be, e.g., crizotinib, alectinib, ceritinib, or a pharmaceutically acceptable salt of any one of these agents. The glutaminase inhibitor may be, e.g., telaglenastat or a pharmaceutically acceptable salt thereof. The JAK inhibitor may be, e.g., tofacitinib, ruxolitinib, upadacitinib, abrocitinib, or a pharmaceutically acceptable salt of any one of these agents. The PLK1 inhibitor may be, e.g., volasertib, onvansertib, rigosertib, BI 2536, or a pharmaceutically acceptable salt of any one of these agents. The Bcl2 inhibitor may be, e.g., venetoclax, navitoclax, obatoclax, or a pharmaceutically acceptable salt of any one of these agents. The HDAC inhibitor may be, e.g., vorinostat, belinostat, panobinostat, romidepsin, practinostat, rocilinostat, quisinostat, abexinostat, resminostat, givinostat, entinostat, mocetinostat, or a pharmaceutically acceptable salt of any one of these agents. The HSP90 inhibitor may be, e.g., onalespib, luminespib, ganetespib, geldanamycin, IPI-504, tanespimycin, alvespimycin, or a pharmaceutically acceptable salt of any one of these agents. The Wnt/β-catenin pathway inhibitor may be, e.g., OMP-18R5, OMP-54F28, OTSA 101, SAH-BCL9, XAV939, IWR1, JW74, J01-017a, PKF115-584, PKF118-310, NCB-0846, LGK974, CWP232291, PRI-724, sulindac, vismodegib, glasdegib, or a pharmaceutically acceptable salt of any one of these agents. The Aurora kinase inhibitor may be, e.g., alisertib, tozasertib, barasertib, danusertib, or a pharmaceutically acceptable salt of any one of these agents. The MDM2 inhibitor may be, e.g., alrizomadlin, idasanutlin, RO5045337, RO5503781, AMG232, CGM097, SAR405838, MK-8242, ALRN-6924, or a pharmaceutically acceptable salt of any one of these agents. The CDK4/6 inhibitor may be, e.g., abemaciclib, ribociclib, palbociclib, or a pharmaceutically acceptable salt of any one of these agents. The YAP/TAZ pathway inhibitor may be, e.g., K-975, TED-347, pazopanib, or a pharmaceutically acceptable salt of any one of these agents. The SOS inhibitor may be, e.g., BI 1701963, BI 3406, BAY-293, or a pharmaceutically acceptable salt of any one of these agents. The Grb2 inhibitor may be, e.g., BP1001, CGP78850, CGP85793, or a pharmaceutically acceptable salt of any one of these agents. The BET inhibitor may be, e.g., ABBV-075, ABBV-744, AZD5153, BAY1238097, CPI-203, CPI-0610, GSK1210151A (or I-BET 151), GSK1324726A (I-BET 726), GSK525762 (or I-BET 762), JQ1, LY294002, MS 436, MS 645, MT-1, olinone, OTX-015, RVX-208, TEN-010, or a pharmaceutically acceptable salt of any one of these agents. The AKT inhibitor may be, e.g., ipatasertib, uprosertib, afuresertib, MK-2206, triciribine, lactoquinomycin, AZD5363, miransertib, capibasertib, or a pharmaceutically acceptable salt of any one of these agents. The MNK inhibitor may be, e.g., ETC-206, SEL-201, BAY1143269, tomivosertib, CGP57380, or a pharmaceutically acceptable salt of any one of these agents. The NTRK inhibitor may be, e.g., entrectinib, larotrectinib, or a pharmaceutically acceptable salt of any one of these agents. The SPH2 inhibitor may be, e.g., JAB-3068, TNO155, SHP099, RMC-4550, IACS-13909, or a pharmaceutically acceptable salt of any one of these agents. The PP2A inhibitor may be, e.g., LB100, cantharidin, cantharidic acid, cytostatin, fostriecin, or a pharmaceutically acceptable salt of any one of these agents. Any of the aforementioned anticancer agents may, in principle, be used in non-salt form or in the form of a pharmaceutically acceptable salt. The present invention specifically and individually relates to each one of the LSD1 inhibitors described herein in combination with each one of the above-described anticancer agents.

As described above, the one or more further therapeutic agents to be used in combination with an LSD1 inhibitor in accordance with the present invention may be (or may comprise) one or more further anticancer agents. Alternatively or in addition, the one or more further therapeutic agents may also comprise an antiemetic agent. Accordingly, the invention also relates to an LSD1 inhibitor for use in the treatment of an NF1-mutant tumor (preferably an NF1-mutant cancer) in combination with one or more further anticancer agents and in combination with an antiemetic agent (and optionally further in combination with radiotherapy); the invention likewise relates to corresponding methods and uses (including all methods and uses described herein above) comprising the combined administration of an LSD1 inhibitor, one or more further anticancer agents, and an antiemetic agent. The antiemetic agent may be, e.g., a 5-HT3 antagonist (or a “setron”), such as, e.g., palonosetron (optionally in combination with netupitant), ramosetron, alosetron, ondansetron, tropisetron, granisetron, dolasetron, azasetron, bemesetron, cilansetron, lerisetron, ricasetron, or zatosetron; olanzapine; a corticosteroid, such as, e.g., methylprednisolone or dexamethasone; or prochlorperazine.

### Articles of Manufacture

The pharmaceutical compositions (or formulations) of the invention can be included in a container, pack or dispenser together with instructions for administration.

Thus, in a further embodiment, the present invention provides an article of manufacture containing an LSD1 inhibitor or a pharmaceutical composition comprising an LSD1 inhibitor for the treatment of an NF1-mutant tumor as described herein.

In some embodiments, the article of manufacture comprises a container and a pharmaceutical composition for use in accordance with the invention as described herein.

In some embodiments, the invention provides an article of manufacture (or a kit) comprising a container and a combination product (as described herein above) for use in the treatment of an NF1-mutant tumor (particularly an NF1-mutant cancer). The invention also provides an article of manufacture (or a kit) comprising (i) a first container comprising the LSD1 inhibitor, (ii) a second container comprising a further anticancer agent (as described above), and (iii) optionally one or more further container(s) comprising one or more further anticancer agent(s), for use in the treatment of an NF1-mutant tumor (particularly an NF1-mutant cancer).

The article of manufacture may further comprise a label or package insert. The term “package insert” is used to refer to instructions customarily included in commercial packages of therapeutic products, that contain information about the indications, usage, dosage, administration, contraindications and/or warnings concerning the use of such therapeutic products. Suitable containers include, for example, blister packs, bottles, vials, syringes, etc. The container may be formed from a variety of materials such as glass or plastic. The container may hold a composition or formulation which is effective for treating the condition and may have a sterile access port (for example, the container may be an intravenous solution bag or a vial having a stopper pierceable by a hypodermic injection needle). The label or package insert indicates that the composition is used for treating the condition of choice, particularly an NF1-mutant tumor. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The article of manufacture or kit may further comprise directions for the combined administration of one or more further anticancer agents (as described above). For example, if the kit comprises a first pharmaceutical composition/formulation comprising the LSD1 inhibitor and a second pharmaceutical composition/formulation comprising a further anticancer agent, the kit may further comprise directions for the simultaneous, sequential or separate administration of the first and the second pharmaceutical compositions/formulations to a subject in need thereof.

In another embodiment, the article of manufacture is suitable for the delivery of solid oral forms of the LSD1 inhibitor, such as tablets or capsules. Such an article of manufacture preferably includes a number of unit dosages. Such articles of manufacture can include a card having the dosages oriented in the order of their intended use. An example of such an article is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, an article of manufacture or kit may comprise: (i) a first container with the LSD1 inhibitor contained therein; (ii) a second container with a further anticancer agent contained therein; and optionally (iii) a third container with a further anticancer agent contained therein, wherein the anticancer agent in the third container is different from the anticancer agent in the second container. Alternatively, or additionally, the kit may comprise another container comprising a pharmaceutically acceptable buffer, such as bacteriostatic water for injection (BWFI), phosphate-buffered saline, Ringer's solution, or dextrose solution. It may further include other materials desirable from a commercial and user standpoint, including other buffers, diluents, filters, needles, and/or syringes.

Where the article of manufacture or kit comprises a composition of the LSD1 inhibitor and a composition of a further anticancer agent, the kit may comprise a container for containing the separate compositions such as, e.g., a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

### Definitions

Unless defined otherwise, all technical and scientific terms used herein have the same meaning as commonly understood by one of ordinary skill in the art to which this invention pertains.

The following definitions apply throughout the present specification and claims, unless specifically indicated otherwise.

A “subject” (or “patient”) for the purposes of the present invention includes both humans and other animals, particularly mammals. Thus, the methods and uses of the invention are applicable to both human therapy and veterinary applications. In preferred embodiments, the subject (or patient) is a mammal (e.g., a human being or a non-human mammal), and most preferably the subject is a human (e.g., a male or female human). A human subject may have any age, including, e.g., 0 to 2 years, 2 to 12 years, 12 to 18 years, or 18 years or more.

The terms “treatment”, “treating” and the like are used herein to generally mean obtaining a desired pharmacological and/or physiological effect. This includes partially or completely curing or ameliorating a disease (such as an NF1-mutant tumor) and/or a symptom or adverse effect attributed to the disease, or partially or completely halting the progression of the disease and/or a symptom or adverse effect attributed to the disease. The term “treatment” as used herein covers any treatment of a disease (such as an NF1-mutant tumor) in a subject and includes, without limitation, inhibiting the disease, i.e., arresting, delaying or slowing down its development/progression; or relieving the disease, i.e., causing its (complete or partial) regression, remission, correction or alleviation. The present invention specifically and distinctly relates to each one of these forms of treatment.

As used herein, the term “therapeutically effective amount” or “effective amount” of a compound (particularly an LSD1 inhibitor) according to the invention refers to an amount sufficient to produce a desired biological effect (e.g., a therapeutic effect or benefit) in a subject. Accordingly, a therapeutically effective amount of a compound may be an amount which is sufficient to treat a disease (such as an NF1-mutant tumor), and/or delay the onset or progression of the disease, and/or alleviate one or more symptoms of the disease, when administered to a subject suffering from or susceptible to that disease. The therapeutically effective amount will vary depending on the compound, the disease state being treated, the severity of the disease treated, the age and relative health of the subject, the route and form of administration, the judgment of the attending medical or veterinary practitioner, and other factors.

The term “pharmaceutically acceptable” denotes an attribute of a material which is useful in preparing a pharmaceutical composition that is generally safe, non-toxic, and neither biologically nor otherwise undesirable and is acceptable for veterinary and/or human pharmaceutical use.

As used herein, a “pharmaceutically acceptable salt” is intended to mean a salt that retains the biological effectiveness of the free acids and/or bases of the specified compound and that is not biologically or otherwise undesirable. A compound may possess one or more sufficiently acidic or sufficiently basic functional groups, or both, and accordingly react with any of a number of inorganic or organic bases, and inorganic or organic acids, to form a pharmaceutically acceptable salt. Exemplary pharmaceutically acceptable salts include those salts prepared by reaction of a compound described herein (particularly an LSD1 inhibitor, such as, e.g., iadademstat), with a mineral or organic acid, such as hydrochlorides, hydrobromides, sulfates, pyrosulfates, bisulfates, sulfites, bisulfites, phosphates, monohydrophosphates, dihydrophosphates, metaphosphates, pyrophosphates, chlorides, bromides, iodides, nitrates, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates (or mesylates), ethane-sulfonates, propanesulfonates, benzenesulfonates (or besylates), toluenesulfonates, trifluoromethansulfonates, naphthalene-1-sulfonates, naphthalene-2-sulfonates, mandelates, pyruvates, stearates, ascorbates, or salicylates. When a compound (particularly an LSD1 inhibitor) carries an acidic moiety, suitable pharmaceutically acceptable salts thereof may include: alkali metal salts, e.g. sodium or potassium salts; alkaline earth metal salts, e.g. calcium or magnesium salts; and salts formed with suitable organic ligands such as ammonia, alkylamines, hydroxyalkylamines, lysine, arginine, N-methylglucamine, procaine and the like. Pharmaceutically acceptable salts are well known in the art (see, e.g., Stahl P H & Wermuth C G (eds.), “Handbook of Pharmaceutical Salts: Properties, Selection, and Use”, Wiley-VCH, 2002 as well as the references cited therein, all of which are incorporated herein by reference).

The terms “pharmaceutical composition” and “pharmaceutical formulation” (or “formulation”) are used interchangeably and denote a mixture or solution comprising a therapeutically effective amount of an active pharmaceutical ingredient (particularly an LSD1 inhibitor) together with one or more pharmaceutically acceptable excipients to be administered to a subject (e.g., a human) in need thereof.

The terms “pharmaceutically acceptable excipient” or “pharmaceutically acceptable carrier” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, lubricants and the like used in formulating pharmaceutical products. They are generally safe for administering to humans according to established governmental standards, including those promulgated by the United States Food and Drug Administration and/or the European Medicines Agency. Pharmaceutically acceptable carriers or excipients are well known to those skilled in the art.

The term “inhibitor”, as used herein, denotes a compound which competes with, decreases, blocks, inhibits, abrogates or interferes in any way with the binding of a particular ligand to a particular receptor or enzyme and/or which decreases, blocks, inhibits, abrogates or interferes in any way with the activity or function of a particular protein, e.g., of a receptor or enzyme.

As used herein, a “small molecule” refers to an organic compound with a molecular weight equal to or below 900 Da (daltons), preferably below 500 Da. The molecular weight is the mass of a molecule and is calculated as the sum of the atomic weights of each constituent element multiplied by the number of atoms of that element in the molecular formula.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

As used herein, the indefinite articles “a” and “an” and the definite article “the” include plural as well as singular referents, unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3 or 4 standard deviations. In certain embodiments, the term “about” or “approximately” means within 25%, 20%, 15%, 10%, 9%, 8%, 7%, 6%, 5%, 4%, 3%, 2%, 1%, 0.5%, 0.1% or 0.05% of a given value or range. Any reference to a numerical value or range provided in connection with the term “about” also includes a reference to the corresponding specific value or range.

Furthermore, it is to be understood that wherever a numerical range is provided/described herein, all values and subranges encompassed by the respective numerical range are specifically provided by the invention. Accordingly, the present invention specifically and individually relates to each value that falls within a numerical range described herein, as well as each and any subrange encompassed by a numerical range described herein.

The present specification describes various compounds by their chemical formulae and their corresponding chemical names. In case of conflict between any chemical formula and the corresponding chemical name indicated herein, the present invention specifically and individually relates to the compound defined by the chemical formula and to the compound defined by the chemical name.

All publications, patent applications, patents, and other references mentioned herein are incorporated by reference in their entirety.

### EXAMPLES

The following examples are provided for illustration of the invention. They should not be considered as limiting the scope of the invention, but merely as being representative thereof.

### Example 1: Effect of LSD1 Inhibitors on NF1-Mutant Tumor Cell Lines

### Experimental Design

Mycoplasma-free tumor cell lines from different tumor types carrying mutations in the NF1 gene (see Table 1) were seeded in 50 μl/well of complete medium (as described in Table 2, all reagents from Thermo Fisher) in 96-well plates at the optimal cell density to ensure log-phase growth throughout the duration of the experiment (see Table 2). The day after seeding, 50 μl of medium containing 9 serial dilutions (1:3) of 2×-concentrated iadademstat (LSD1 inhibitor; used as the dihydrochloride salt) were added to the cells to obtain 100 μl of cells treated with 1×-concentrated compound at each dilution. Additionally, the effect of other LSD1 inhibitors, i.e. bomedemstat (bis-tosylate salt) and pulrodemstat (besylate salt), was likewise evaluated in a subset of these tumor cell lines (sNF96.2, sNF02.2, MOLM13 and NCIH510A).

Each experimental condition was tested in technical triplicates, including medium-only wells and vehicle-treated controls for background correction and normalization, respectively. After treatment, cells were incubated at 37° C. in a humidified and controlled 5% CO₂ atmosphere (see Table 2 for duration of treatment according to the growth curve of each cell line and number of cells seeded) and compound and medium refreshment was performed by adding 50 μl of medium supplemented with 1×-concentrated compound at each corresponding dilution when the duration of the treatment exceeded 4 days of drug incubation (see Table 2). After treatment, cell viability was evaluated using either the MTT assay (Sigma-Aldrich) or AlamarBlue™ Cell viability reagent (Life Technologies), following manufacturer's instructions. Background was calculated as the mean of the values of medium-only controls and subtracted from each data point. The average of background-corrected technical triplicates was calculated and normalized by the mean of vehicle-treated controls (corresponding to 100% of viability). Data were analyzed using GraphPad PRISM® version 9.0.1 (GraphPad Software, Inc., La Jolla, CA/USA) to calculate the best-fitting curves and the EC₅₀ values (corresponding to the concentration of compound at which a half (50%) maximal effect is obtained; lower EC₅₀ values thus indicate greater potency).

TABLE 1
Different tumor cell lines used and their corresponding NF1 mutations (SCLC: small-cell lung cancer;
MPNST: malignant peripheral nerve sheath tumor; ALL: acute lymphocytic leukemia; AML: acute myeloid
leukemia; TN breast cancer: triple-negative breast cancer; LOH: loss of heterozygosity; p.: mutation
in protein sequence; c.: mutation in DNA sequence; SNP: single nucleotide polymorphism; SNV: single
nucleotide variant; *: mutation in 3′ generating STOP codon; NS: not specified).

| | | | NF1 mutation variant |
|---|---|---|---|
| Tumor type | Cell line | NF1 mutation position | classification |
| SCLC | NCIH510A | p. D2184G/p. L2618L | Missense Mutation/Silent |
| Plexiform neurofibroma | hTERT ipnNF95.11c | c. 1756delACTA | Deletion |
| MPNST | sNF96.2 | c. 3683delC | Frameshift mutation + LOH |
| ALL | MOLT4 | p. G1502G/ NS | Splice site/silent |
| AML | MOLM13 | p. E1720D | Missense Mutation |
| Rhabdoid tumor | A204 | p. L1274F | Missense Mutation |
| Plexiform neurofibroma | hTERT ipNF95.11b C | c. 1756delACTA/ LOH | Deletion + LOH |
| MPNST | sNF02.2 | c. 4868A > T | Missense Mutation |
| TN breast cancer | MDAMB231 | INS p. T467fs | Frame Shift Insertion |
| Ewing's sarcoma | MHHES1 | p. P1951Q | Missense Mutation |
| Ewing's sarcoma | TC71 | SNP NS | Splice Site |
| Burkitt's lymphoma | DAUDI | p. K1385R/ 27 additional silent SNPs | Missense Mutation/Silent |
| Rhabdomyosarcoma | RD | p. E977* | Nonsense Mutation |
| SCLC | NCIH446 | p. D2346H | Missense Mutation |
| MPNST | MPNST-NF1-18b | SNV at intron 14 | Splice Site + LOH |

TABLE 2
Cell lines used and their corresponding experimental conditions
for iadademstat treatment (FBS: fetal bovine serum).

| | | | | Iadademstat | |
|---|---|---|---|---|---|
| | | | | | Medium + |
| | | | | | compound |
| | | | | Treatment | refreshment |
| Tumor type | Cell line | Cells/well | Medium | (Days) | (Day) |
| SCLC | NCIH510A | 3000 | RPMI-1640, | 10 | 6 |
| | | | 2 mM L-glutamine, | | |
| | | | 10% FBS | | |
| Plexiform | hTERT | 250 | DMEM high glucose, | 7 | 5 |
| neurofibroma | ipnNF95.11c | | 2 mM L-glutamine, | | |
| | | | 10% FBS | | |
| MPNST | sNF96.2 | 8000 | DMEM high glucose, | 6 | 3 |
| | | | 1X GlutaMAX ™, | | |
| | | | 1X sodium pyruvate, | | |
| | | | 10% FBS | | |
| ALL | MOLT4 | 4000 | RPMI-1640, | 10 | 6 |
| | | | 2 mM L-glutamine, | | |
| | | | 10% FBS | | |
| AML | MOLM13 | 4000 | RPMI-1640, | 4 | — |
| | | | 2 mM L-glutamine, | | |
| | | | 10% FBS | | |
| Rhabdoid | A204 | 900 | McCoy's 5A, | 6 | 3 |
| tumor | | | 2 mM L-glutamine, | | |
| | | | 10% FBS | | |
| Plexiform | hTERT | 500 | DMEM high glucose, | 7 | 5 |
| neurofibroma | ipNF95.11b | | 2 mM L-glutamine, | | |
| | C | | 10% FBS | | |
| MPNST | sNF02.2 | 1000 | DMEM high glucose, | 6 | 3 |
| | | | 1X GlutaMAX ™, | | |
| | | | 1X sodium pyruvate, | | |
| | | | 10% FBS | | |
| TN breast | MDAMB231 | 8000 | DMEM high glucose, | 4 | — |
| cancer | | | 2 mM L-glutamine, | | |
| | | | 10% FBS | | |
| Ewing's | MHHES1 | 2000 | RPMI-1640, | 6 | 3 |
| sarcoma | | | 2 mM L-glutamine, | | |
| | | | 10% FBS | | |
| Ewing's | TC71 | 1000 | DMEM high glucose, | 6 | 3 |
| sarcoma | | | 2 mM L-glutamine, | | |
| | | | 10% FBS | | |
| Burkitt's | DAUDI | 2000 | RPMI-1640, | 4 | — |
| lymphoma | | | 2 mM L-glutamine, | | |
| | | | 10% FBS | | |
| Rhabdomyosarcoma | RD | 1000 | DMEM high glucose, | 6 | 3 |
| | | | 2 mM L-glutamine, | | |
| | | | 10% FBS | | |
| SCLC | NCIH446 | 1000 | RPMI-1640, | 10 | 5 |
| | | | 2 mM L-glutamine, | | |
| | | | 10% FBS | | |
| MPNST | MPNST- | 500 | DMEM high glucose, | 6 | 3 |
| | NF1-18b | | 1X GlutaMAX ™, | | |
| | | | 1X sodium pyruvate, | | |
| | | | 10% FBS | | |

### Results

Fifteen NF1-mutant cell lines representing ten different tumor types were used to evaluate the effects of the LSD1 inhibitor iadademstat on cell viability in a range of different NF1-mutant tumors.

LSD1 inhibitors such as iadademstat have been reported to exert their therapeutic effect by inducing cancer cell differentiation and inhibiting cancer cell proliferation rather than by killing cancer cells (Sacilotto N et al., ACS Pharmacol Transl Sci, 2021, 4 (6): 1818-34, doi: 10.1021/acsptsci.1c00223). In line with this, a tumor cell viability reduction of more than 30% reflects a potent therapeutic effect of the corresponding LSD1 inhibitor. In the present experiments, the response obtained after LSD1 inhibitor treatment was therefore classified into the following three groups: (1) strong response (viability reduction>30%); (2) medium response (viability reduction>15% and <30%); (3) low response (viability reduction<15%). Table 3 summarizes the EC₅₀ values obtained for iadademstat together with the classification according to viability reduction after LSD1 inhibitor treatment.

TABLE 3
Effects of the LSD1 inhibitor iadademstat on cell viability
of the indicated NF1-mutant tumor cell lines.

| | | Iadademstat | |
|---|---|---|---|
| Tumor type | Cell line | EC₅₀ (nM) | Response |
| SCLC | NCIH510A | 0.160 | Strong |
| Plexiform | hTERT | 0.841 | |
| neurofibroma | ipnNF95.11c | | |
| MPNST | sNF96.2 | 0.042 | |
| ALL | MOLT4 | 0.795 | |
| AML | MOLM13 | 0.158 | |
| Rhabdoid | A204 | 0.100 | |
| tumor | | | |
| MPNST | MPNST- | 0.025 | |
| | NF1-18b | | |
| Plexiform | hTERT | 0.140 | Medium |
| neurofibroma | ipNF95.11b | | |
| | C | | |
| MPNST | sNF02.2 | 0.020 | |
| TN breast | MDAMB231 | Not determined | Low |
| cancer | | | |
| Ewing's | MHHES1 | Not determined | |
| sarcoma | | | |
| Ewing's | TC71 | Not determined | |
| sarcoma | | | |
| Burkitt's | DAUDI | Not determined | |
| lymphoma | | | |
| Rhabdomyosarcoma | RD | Not determined | |
| SCLC | NCIH446 | Not determined | |

As shown in Table 3, 9 out of 15 tumor cell lines (60.0%) carrying NF1 mutations responded advantageously well to LSD1 inhibitor treatment. Among these 9 cell lines, 7 (46.7%) displayed a strong response and 2 (13.3%) displayed a medium response to the LSD1 inhibitor iadademstat. Notably, all 9 advantageously well responding cell lines displayed subnanomolar EC₅₀ values for iadademstat (see Table 3), which points at a clinically relevant therapeutic effect even when administered at very low doses.

The effect of LSD1 inhibitors on cell viability of NF1-mutant tumor cells was further tested using 2 additional LSD1 inhibitors, namely bomedemstat and pulrodemstat. Bomedemstat, like iadademstat, is an irreversible LSD1 inhibitor, whereas pulrodemstat is a reversible LSD1 inhibitor. Cell viability was evaluated in sNF96.2 (MPNST), sNF02.2 (MPNST), MOLM13 (AML) and NCIH510A (SCLC) cell lines as per the method described above (see Table 2). The results thus obtained, as presented in Table 4 (and, for jadademstat, in Table 3 above), clearly show that all three LSD1 inhibitors are effective against NF1-mutant tumors, irrespective of whether they are irreversible or reversible LSD1 inhibitors.

TABLE 4
Effect of different LSD1 inhibitors on cell viability
of the indicated NF1-mutant tumor cell lines.

| | Cell line | | | |
|---|---|---|---|---|
| | sNF96.2 | sNF02.2 | MOLM13 | NCIH510A |
| LSD1 inhibitor | EC₅₀ (nM) | EC₅₀ (nM) | EC₅₀ (nM) | EC₅₀ (nM) |
| Bomedemstat | 1.277 | 0.094 | 5.380 | 10.510 |
| Pulrodemstat | 0.261 | 0.166 | 0.820 | 1.734 |

Among the LSD1 inhibitors tested, iadademstat displayed the highest potency (lowest EC₅₀) in all four NF1-mutant tumor cell lines.

These results show that LSD1 inhibitors, including both irreversible LSD1 inhibitors (such as iadademstat and bomedemstat) as well as reversible LSD1 inhibitors (such as pulrodemstat), are advantageously effective in the treatment of NF1-mutant tumors, with both germline and somatic NF1 mutations.

While the invention has been described in connection with specific embodiments thereof, it will be understood that it is capable of further modifications and this patent or patent application is intended to cover any variations, uses or adaptations of the invention following, in general, the principles of the invention and including such departures from the present disclosure as come within known or customary practice within the art to which the invention pertains and as may be applied to the essential features hereinbefore set forth and as follows in the appended claims.

1. An LSD1 inhibitor for use in the treatment of an NF1-mutant tumor.

2. A pharmaceutical composition comprising an LSD1 inhibitor and optionally one or more pharmaceutically acceptable excipients for use in the treatment of an NF1-mutant tumor.

3. A method of treating an NF1-mutant tumor in a subject in need thereof, comprising administering a therapeutically effective amount of an LSD1 inhibitor to the subject.

4. Use of an LSD1 inhibitor for the treatment of an NF1-mutant tumor.

5. Use of an LSD1 inhibitor for the preparation of a pharmaceutical composition for the treatment of an NF1-mutant tumor.

6. The LSD1 inhibitor for use according to claim 1, the pharmaceutical composition for use according to claim 2, the method of claim 3, or the use of claim 4 or 5, wherein the LSD1 inhibitor is a small molecule.

7. The LSD1 inhibitor for use according to claim 1 or 6, the pharmaceutical composition for use according to claim 2 or 6, the method of claim 3 or 6, or the use of any one of claims 4 to 6, wherein the LSD1 inhibitor is selected from the group consisting of iadademstat, pulrodemstat, bomedemstat, seclidemstat, 1-((4-(methoxymethyl)-4-(((1R,2S)-2-phenylcyclopropylamino)methyl) piperidin-1-yl)methyl)cyclobutanecarboxylic acid, 3-(cyanomethyl)-3-(4-{[(1R,2S)-2-phenylcyclopropyl]amino}piperidin-1-yl) azetidine-1-sulfonamide, 4-[5-[(3S)-3-aminopyrrolidine-1-carbonyl]-2-[2-fluoro-4-(2-hydroxy-2-methyl-propyl)phenyl]phenyl]-2-fluoro-benzonitrile, and pharmaceutically acceptable salts thereof.

8. The LSD1 inhibitor for use according to any one of claims 1, 6 or 7, the pharmaceutical composition for use according to any one of claims 2, 6 or 7, the method of any one of claims 3, 6 or 7, or the use of any one of claims 4 to 7, wherein the LSD1 inhibitor is selected from the group consisting of iadademstat, pulrodemstat, bomedemstat, and pharmaceutically acceptable salts thereof.

9. The LSD1 inhibitor for use according to any one of claims 1 or 6 to 8, the pharmaceutical composition for use according to any one of claims 2 or 6 to 8, the method of any one of claims 3 or 6 to 8, or the use of any one of claims 4 to 8, wherein the LSD1 inhibitor is iadademstat or a pharmaceutically acceptable salt thereof.

10. The LSD1 inhibitor for use according to claim 9, the pharmaceutical composition for use according to claim 9, the method of claim 9, or the use of claim 9, wherein the LSD1 inhibitor is iadademstat dihydrochloride.

11. The LSD1 inhibitor for use according to any one of claims 1 or 6 to 8, the pharmaceutical composition for use according to any one of claims 2 or 6 to 8, the method of any one of claims 3 or 6 to 8, or the use of any one of claims 4 to 8, wherein the LSD1 inhibitor is pulrodemstat or a pharmaceutically acceptable salt thereof.

12. The LSD1 inhibitor for use according to claim 11, the pharmaceutical composition for use according to claim 11, the method of claim 11, or the use of claim 11, wherein the LSD1 inhibitor is pulrodemstat besylate.

13. The LSD1 inhibitor for use according to any one of claims 1 or 6 to 8, the pharmaceutical composition for use according to any one of claims 2 or 6 to 8, the method of any one of claims 3 or 6 to 8, or the use of any one of claims 4 to 8, wherein the LSD1 inhibitor is bomedemstat or a pharmaceutically acceptable salt thereof.

14. The LSD1 inhibitor for use according to claim 13, the pharmaceutical composition for use according to claim 13, the method of claim 13, or the use of claim 13, wherein the LSD1 inhibitor is bomedemstat bis-tosylate.

15. The LSD1 inhibitor for use according to any one of claims 1 or 6 to 14, the pharmaceutical composition for use according to any one of claims 2 or 6 to 14, the method of any one of claims 3 or 6 to 14, or the use of any one of claims 4 to 14, wherein the NF1-mutant tumor is an NF1-mutant cancer.

16. The LSD1 inhibitor for use according to any one of claims 1 or 6 to 14, the pharmaceutical composition for use according to any one of claims 2 or 6 to 14, the method of any one of claims 3 or 6 to 14, or the use of any one of claims 4 to 14, wherein the NF1-mutant tumor is selected from NF1-mutant leukemia, NF1-mutant lymphoma, NF1-mutant lung cancer, NF1-mutant breast cancer, NF1-mutant esophagogastric cancer, NF1-mutant esophageal cancer, NF1-mutant gastric cancer, NF1-mutant gastrointestinal cancer, NF1-mutant colorectal cancer, NF1-mutant liver cancer, NF1-mutant ovarian cancer, NF1-mutant uterine cancer, NF1-mutant cervical cancer, NF1-mutant pancreatic cancer, NF1-mutant prostate cancer, NF1-mutant bladder cancer, NF1-mutant pheochromocytoma, NF1-mutant head and neck cancer, NF1-mutant neuroblastoma, NF1-mutant glioblastoma, NF1-mutant optic pathway glioma, NF1-mutant skin cancer, NF1-mutant malignant rhabdoid tumor, NF1-mutant rhabdomyosarcoma, NF1-mutant Ewing sarcoma, NF1-mutant malignant peripheral nerve sheath tumor, NF1-mutant plexiform neurofibroma, NF1-mutant ganglioneuroma, NF1-mutant Lisch nodule, and NF1-mutant cutaneous neurofibroma.

17. The LSD1 inhibitor for use according to any one of claims 1 or 6 to 16, the pharmaceutical composition for use according to any one of claims 2 or 6 to 16, the method of any one of claims 3 or 6 to 16, or the use of any one of claims 4 to 16, wherein the NF1-mutant tumor is NF1-mutant leukemia.

18. The LSD1 inhibitor for use according to claim 17, the pharmaceutical composition for use according to claim 17, the method of claim 17, or the use of claim 17, wherein the NF1-mutant leukemia is NF1-mutant acute myeloid leukemia or NF1-mutant acute lymphocytic leukemia.

19. The LSD1 inhibitor for use according to any one of claims 1 or 6 to 16, the pharmaceutical composition for use according to any one of claims 2 or 6 to 16, the method of any one of claims 3 or 6 to 16, or the use of any one of claims 4 to 16, wherein the NF1-mutant tumor is NF1-mutant lung cancer.

20. The LSD1 inhibitor for use according to claim 19, the pharmaceutical composition for use according to claim 19, the method of claim 19, or the use of claim 19, wherein the NF1-mutant lung cancer is NF1-mutant small-cell lung cancer.

21. The LSD1 inhibitor for use according to any one of claims 1 or 6 to 16, the pharmaceutical composition for use according to any one of claims 2 or 6 to 16, the method of any one of claims 3 or 6 to 16, or the use of any one of claims 4 to 16, wherein the NF1-mutant tumor is NF1-mutant malignant rhabdoid tumor.

22. The LSD1 inhibitor for use according to any one of claims 1 or 6 to 16, the pharmaceutical composition for use according to any one of claims 2 or 6 to 16, the method of any one of claims 3 or 6 to 16, or the use of any one of claims 4 to 16, wherein the NF1-mutant tumor is NF1-mutant malignant peripheral nerve sheath tumor.

23. The LSD1 inhibitor for use according to any one of claims 1 or 6 to 14, the pharmaceutical composition for use according to any one of claims 2 or 6 to 14, the method of any one of claims 3 or 6 to 14, or the use of any one of claims 4 to 14, wherein the NF1-mutant tumor is NF1-mutant plexiform neurofibroma.

24. The LSD1 inhibitor for use according to any one of claims 1 or 6 to 23, the pharmaceutical composition for use according to any one of claims 2 or 6 to 23, the method of any one of claims 3 or 6 to 23, or the use of any one of claims 4 to 23, wherein the NF1-mutant tumor has one or more inactivating mutations or inactivating genetic alterations affecting the NF1 gene.

25. The LSD1 inhibitor for use according to any one of claims 1 or 6 to 24, the pharmaceutical composition for use according to any one of claims 2 or 6 to 24, the method of any one of claims 3 or 6 to 24, or the use of any one of claims 4 to 24, wherein the LSD1 inhibitor or the pharmaceutical composition is administered to a subject having neurofibromatosis type I.

26. The LSD1 inhibitor for use according to any one of claims 1 or 6 to 24, the pharmaceutical composition for use according to any one of claims 2 or 6 to 24, the method of any one of claims 3 or 6 to 24, or the use of any one of claims 4 to 24, wherein the LSD1 inhibitor or the pharmaceutical composition is administered to a subject not having neurofibromatosis type I.

27. The LSD1 inhibitor for use according to any one of claims 1 or 6 to 26, the pharmaceutical composition for use according to any one of claims 2 or 6 to 26, the method of any one of claims 3 or 6 to 26, or the use of any one of claims 4 to 26, wherein the LSD1 inhibitor or the pharmaceutical composition is administered to a subject which is a human.

28. The LSD1 inhibitor for use according to any one of claims 1 or 6 to 27, the pharmaceutical composition for use according to any one of claims 2 or 6 to 27, the method of any one of claims 3 or 6 to 27, or the use of any one of claims 4 to 27, wherein the LSD1 inhibitor or the pharmaceutical composition is administered orally.

29. The LSD1 inhibitor for use according to any one of claims 1 or 6 to 28, the pharmaceutical composition for use according to any one of claims 2 or 6 to 28, the method of any one of claims 3 or 6 to 28, or the use of any one of claims 4 to 28, wherein the LSD1 inhibitor or the pharmaceutical composition is administered in combination with one or more further anticancer agents and/or in combination with radiotherapy.

30. An article of manufacture comprising, in the same pharmaceutical formulation or in separate pharmaceutical formulations, an LSD1 inhibitor and one or more further anticancer agents, for use in the treatment of an NF1-mutant tumor.

31. A method of treating an NF1-mutant tumor in a subject in need thereof, comprising administering to the subject a therapeutically effective amount of an article of manufacture comprising, in the same pharmaceutical formulation or in separate pharmaceutical formulations, an LSD1 inhibitor and one or more further anticancer agents.

32. The article of manufacture for use according to claim 30, or the method of claim 31, wherein:
the LSD1 inhibitor is as defined in any one of claims 6 to 14; and/or
the NF1-mutant tumor is as defined in any one of claims 15 to 24; and/or
the subject to whom the article of manufacture is administered is as defined in any one of claims 25 to 27; and/or
the article of manufacture is administered orally.
